(19) United States
(12) Patent Application Publication
(10) Pub. No.: US 2022/0334097 A1
(43) Pub. Date: Oct. 20, 2022

(54) System for Sensing a Molecule

(71) Applicant: Northeastern University, Boston, MA (US)

(72) Inventors: Meni Wanunu, Sharon, MA (US); Mohammadamin Alibakhshi, Boston, MA (US); Fatemeh Farhangdoust, Allston, MA (US)

(21) Appl. No.: 17/754,114

(22) PCT Filed: Sep. 25, 2020
(86) PCT No.: PCT/US2020/052922

§ 371 Date: Mar. 24, 2022

Related U.S. Application Data
(60) Provisional application No. 62/906,213, filed on Sep. 26, 2019.

(51) Int. Cl. G01N 33/487; G01N 21/64

(52) U.S. Cl. CPC G01N 33/48721; G01N 21/6452

(57) ABSTRACT

A system and apparatus are provided for sensing a molecule in a sample for identifying the molecule in the sample. Also provided is a method of manufacturing an apparatus for sensing a molecule in a sample. The system and apparatus may contain a composition of a mixture of a buffer solution and a sample solution containing a sample. The apparatus contains a series of wells. The sample is deposited into the system and a molecule having an electric charge in the sample attaches to a layer in a well of the apparatus, allowing for a sensor connected to the apparatus to identify the molecule.

### RELATED APPLICATION

This application claims the benefit of U.S. Provisional Application No. 62/906,213, filed on Sep. 26, 2019. The entire teachings of the above application are incorporated herein by reference.

### GOVERNMENT SUPPORT

This invention was made with government support under 1R01HG009186 from The National Institutes of Health. The government has certain rights in the invention.

### BACKGROUND

Over the past 20 years, DNA sequencing technologies have undergone tremendous improvements in throughput and quality. One advantage of single-molecule DNA sequencing technologies is that long DNA molecules can be read in a single run, which greatly facilitates genome assembly and aids in the construction of reference-quality genomes. A version of single-molecule sequencing called single molecule, real-time (SMRT) sequencing, replicates DNA by a polymerase and is monitored in real-time by tracking the incorporation of fluorescently labeled nucleotides of different colors. This method offers long reads (>15,000 bp) and the ability to detect epigenetic modifications by tracking delays in the polymerase kinetics of incorporation of different nucleotides. Nonetheless, while the throughput and accuracy of SMRT-sequencing has vastly improved in recent years, loading efficiency and length bias are both still problematic. This translates to high DNA input requirements (>100 ng) and length-purified libraries in order to alleviate bias toward reading short DNA lengths.

### SUMMARY OF THE INVENTION

Described herein are example embodiments of a new format for zero-mode waveguides (ZMWs), which are optical cavities for single-molecule fluorescence detection commercially used for DNA sequencing applications. In this format, the waveguides contain built-in electrodes at their bottoms, which allows a voltage to be applied across the ZMWs, thereby generating electric fields that capture biomolecules efficiently. Application of a voltage to the electrode layer with the use of proper electrolyte allows efficient electrophoretic DNA capture at picogram levels. To do so, a thin metallic film (M_{Lasso}) is deposited on a substrate (S), and in operation the thin metallic film serves as an electrode. In some embodiments, the electrode is insulated from a metallic ZMW layer (M_{ZMW}) using a dielectric spacer (D). This device eliminates the need for free-standing membranes and, therefore, is mechanically stable, allows scaled-up fabrication, reduces background optical noise, and improves DNA loading efficiency by several orders of magnitude. Dual-ring, metallic, zero-mode waveguides have been previously used in the context of electrochemical analysis, wherein electrochemical signals are amplified by a repetition of reduction and oxidation reaction at two closely located electrodes. However, this idea has never been used for DNA/RNA sequencing and sensing applications.

Following that general description of embodiments of the invention, below are some particular example embodiments.

In an embodiment, an apparatus for sensing a molecule comprises a substrate layer, a sample interface layer, and an electrically conductive layer. The sample interface layer has a sample interface side and a substrate layer facing side. The electrically conductive layer is disposed between the substrate layer facing side of the sample interface layer and the substrate layer. The sample interface layer and the electrically conductive layer each define a respective opening therethrough that, aligned, compose a wall of a well with a bottom boundary defined by the substrate layer. The electrically conductive layer, when energized with a given polarity relative to an electrically conductive element in a sample at the sample interface layer, produces an electric field from the electrically conductive layer through the well to the electrically conductive element. The electric field is sufficient to draw a molecule, of polarity opposite the given polarity, from the sample at the sample interface side through the well toward the electrically conductive layer.

In an embodiment, a method of manufacturing an apparatus for detecting a molecule comprises applying a negative beam lithography coating to a substrate layer, removing portions of the electron beam lithography coating to define a pattern of support elements, forming an electrically conductive layer above the substrate layer within the pattern of support elements, forming a sample interface layer above the electrically conductive layer within the pattern of support elements, and removing the support elements to create wells having a boundary defined by the electrically conductive layer and the sample interface layer.

In an embodiment, a system for sensing a molecule comprises a structure, a power supply and a molecule sensor. The structure includes a sample interface layer, an electrically conductive layer, and a substrate layer, arranged in that order. The sample interface layer and electrically conductive layer define openings therethrough that are aligned to form a well from a sample interface side of the sample interface layer to the substrate layer. The power supply is coupled to the electrically conductive layer and at least one electrically conductive element. The at least one electrically conductive element is located externally from the well. The electrically conductive layer and the electrically conductive element produce an electric field in the well of sufficient strength to cause a molecule, from a sample at the sample interface side of the sample interface layer, with a polarity opposite a polarity of the electrically conductive layer, to travel via the well toward the electrically conductive layer. The molecule sensor is communicatively coupled to the structure in an arrangement that enables sensing of the molecule in the well. A controller is configured to enable the power supply to deliver a voltage difference to the electrically conductive layer and at least one electrically conductive element and to provide an indicator signal to the molecule sensor to notify the molecule sensor to perform a sensing of the molecule.

In an embodiment, a method of sensing a molecule comprises applying an electric field to a sample in proximity of a structure defining a well and sensing the molecule within the well. The electric field applied to the sample is of sufficient strength to cause a molecule of a given polarity in the sample to be drawn toward a surface in the well of an opposite polarity.

In an embodiment of the invention, an electrically actuatable ZMW is configured for high-throughput pg-level sequencing.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A is a diagram of a user at a molecule sensing station, in accordance with an embodiment of the invention.

FIG. 1B is a schematic diagram of a molecule sensing chamber located in a sample holding assembly, in accordance with an embodiment of the invention.

FIG. 1C is a schematic diagram of a molecule sensor coupled to a controller, the controller also being coupled to a power source, in accordance with an embodiment of the invention.

FIG. 2A is a mechanical diagram of an apparatus including a sample interface layer, an electrically nonconductive layer, an electrically conductive layer, and a substrate layer, arranged to define a well, in accordance with an embodiment of the invention.

FIG. 2B is mechanical diagram of an apparatus having a sample interface layer, an electrically nonconductive layer, an electrically conductive layer, to which a primer-enzyme complex is coupled, and a substrate, in accordance with an embodiment of the invention.

FIG. 3A-F depict a schematic diagram of the method of manufacturing a molecule sensing chamber for detecting a molecule, in accordance with an embodiment of the invention.

FIG. 3G is an illustration of the manufactured molecule sensing chamber of the embodiment of FIG. 3A.

FIGS. 4A-B depict a molecule sensing apparatus to which a voltage has not been applied, in accordance with an embodiment of the invention.

FIGS. 4C-D depict a molecule sensing apparatus to which a voltage has been applied, in accordance with an embodiment of the invention.

FIG. 5A is a side view of a well and layers defining the well, in accordance with an embodiment of the invention.

FIG. 5B is a top view of a molecule sensing apparatus, in accordance with an embodiment of the invention.

### DETAILED DESCRIPTION

A description of example embodiments follows.

“Electrically conductive element” is defined herein as a material having a significant degree of electrical conductivity that is configured to function in the presence of solids, liquids and gases.

“Electrically conductive layer” is defined herein as a layer of a material having a low resistivity that enables organic matter and allows for adhesion of the organic matter without affecting the organic matter's functioning.

“Non-conductive layer” is defined herein as a layer of material that has a high resistivity that insulates a sample interface layer from an electrically conductive layer.

“Organic matter” is defined herein as a biological material that facilitates the binding or adhesion of a molecule, or a biological material that facilitates a reaction of which the molecule is a component.

“Sample interface layer” is defined herein as a zero-mode waveguide metallic layer with which the sample interacts. In one embodiment, the sample interface layer is a zero-mode waveguide metallic layer.

“Substrate layer” is defined herein as a solid transparent layer through which a sensor signal can pass to sense a molecule. In one embodiment, the substrate layer is a solid transparent layer through which visible or near-infrared light travels.

“Substrate layer facing side” is defined herein as a side of the sample interface layer that is in contact with the electrically conductive or non-conductive layer.

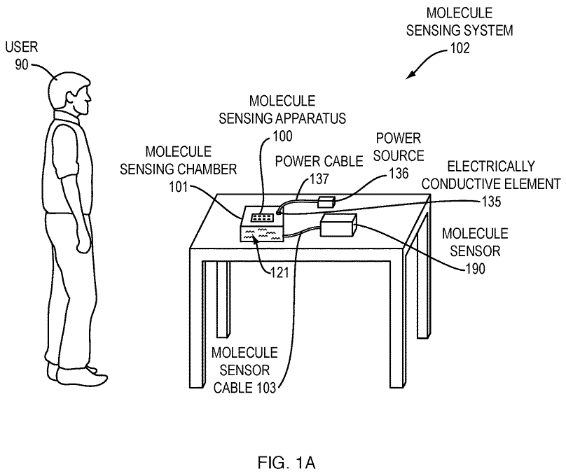

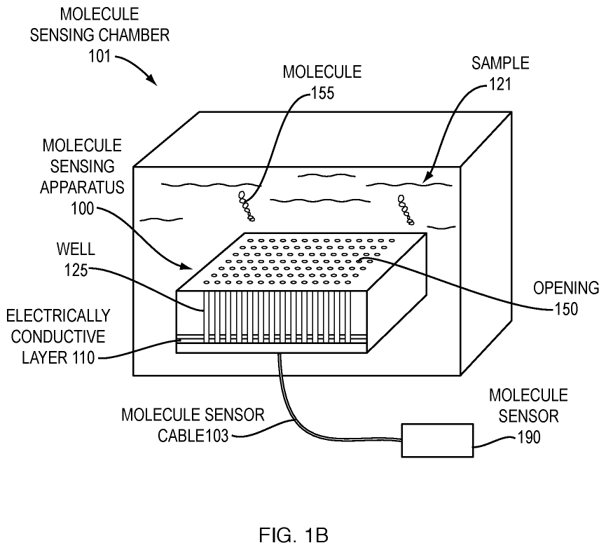

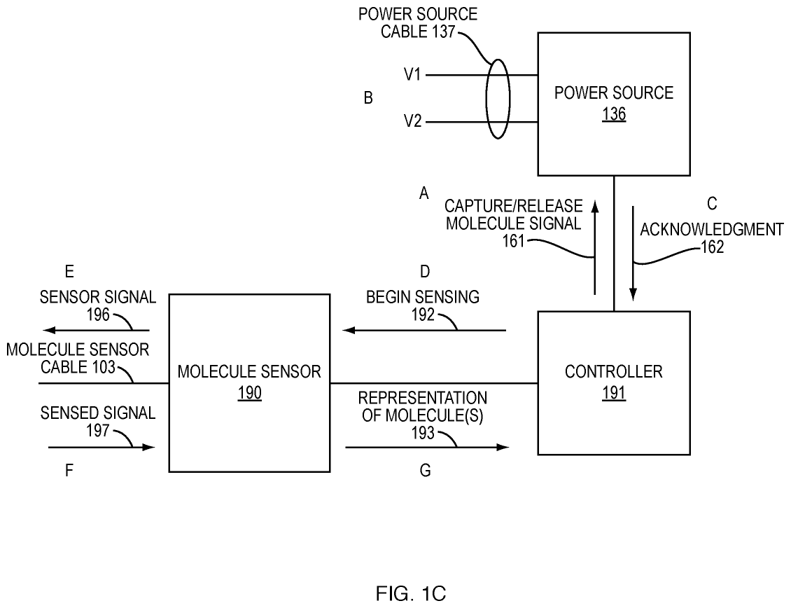

FIG. 1A depicts a user 90 in front of a molecule sensing system 102 that comprises a molecule sensing chamber 101 with molecule sensing apparatus 100, power source 136 (also referred to herein as a power supply), and molecule sensor 190, in accordance with an embodiment of the invention.

The molecule sensing chamber 101 is connected to the power source 136 through a power cable 137. The molecule sensing chamber 101 is connected to the molecule sensor 190 through a molecule sensor cable 103. The power source 136 may have a reference terminal (not shown) that is floating or grounded, and the reference terminal is electrically coupled to an electrically conductive element 135 via the power cable 137. The power supply 136 also includes a power terminal (not shown) that is electrically coupled to the molecule sensing apparatus 100 via the power cable 137.

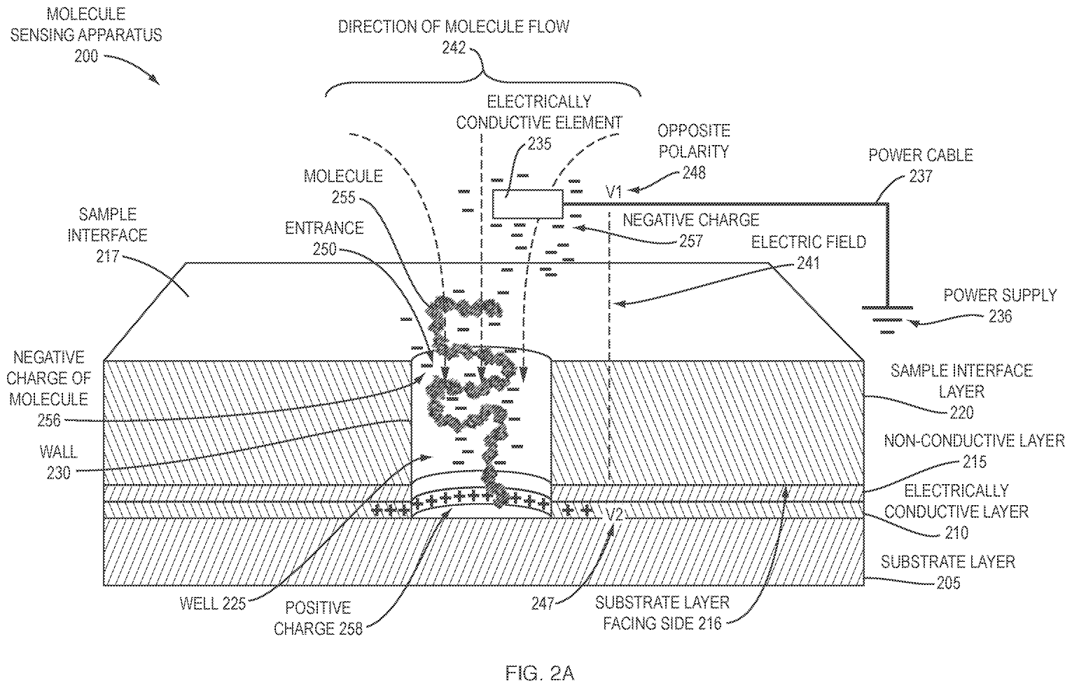

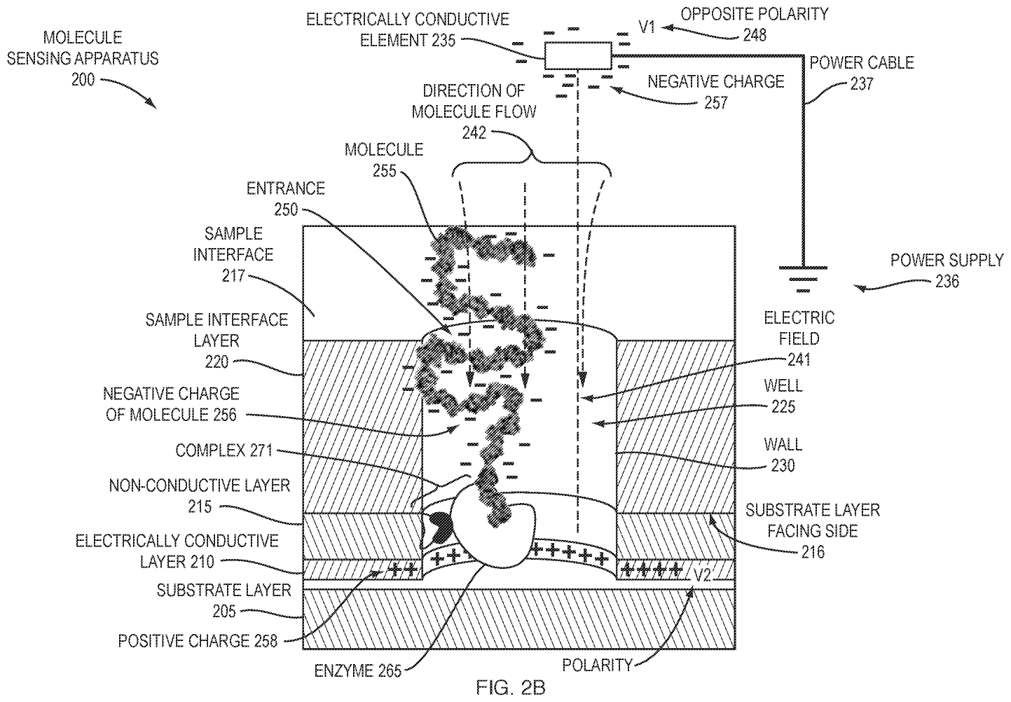

Through operation of the molecule sensing system 102, as described in detail below, the user 90 can determine whether a molecule, not shown in FIG. 1A but illustrated in FIG. 2A-B as molecule 255, is present within a fluidic sample 121, as depicted in FIG. 1, contained in the molecule sensing chamber 101. Once the sample 121 is located in the molecule sensing chamber 101 and the power source 136 is activated, the molecule in the sample is drawn into a well (not shown) of the molecule sensing apparatus 100 via an electric field (not shown) that is generated by the electrically conductive element 135 in combination with a feature of the molecule sensing apparatus 100, as illustrated in FIG. 1B. The electric field is depicted in detail in FIG. 2A-B as electric field 241.

FIG. 1B depicts the molecule sensing chamber 101 and the molecule sensing apparatus 100. Located in the molecule sensing chamber 101 is the fluidic sample 121 and the molecule sensing apparatus 100. Above the molecule sensing apparatus 100 is the electrically conductive element 135 coupled to the power source 136 via the power cable 137. During operation, the electrically conductive element 136 has a voltage level different from the voltage level of an electrically conductive layer 110 in the molecule sensing apparatus 100. The voltage that is more positive relative to the other is said to have positive polarity, and vice-versa. Thus, the electrically conductive element 135 has a polarity that is opposite a polarity of an electrically conductive layer 110 in the molecule sensing apparatus 100, where the electrically conductive layer is partially defining a well 125. The polarity (i.e., electrically positive or negative) of the electrically conductive element 135 may be the same as the polarity of the molecule 155 in the sample 121. The electric field (not shown) created by the two voltages causes a molecule 155 in the sample 121 of opposite polarity from the polarity of the electrically conductive layer 110 to be drawn to the electrically conductive layer 110 through an entrance 150 of a given well 125.

The molecule sensor 190 is coupled to the molecule sensing chamber 101 via the molecule sensor cable 103, which projects a visible or near-infrared light 191 into the molecule sensing chamber 101 and into the wells 125 via a substrate 105 of the molecule sensing apparatus 100 for sensing the presence of the molecule in the given well 125. As will be described more hereinbelow, the orientation of projection of the light 191 into the wells 125, in combination with features of the molecule sensing apparatus 100, provide sensing of the molecule 155 with low noise from the sample 121.

More particularly, embodiments disclosed herein allow for the capture and detection of small amounts of nucleic acid that do not require amplification. Existing methods that use free-standing membranes have drawbacks, such as difficulty to manufacture, fragility, and optical background noise. The present disclosure describes a new non-freestanding structure that minimizes optical background noise and uses an electric field to draw nucleic acid molecules into a space where they are then analyzed using a molecule sensor system. The present disclosure also describes a molecule sensing system that utilizes AC and DC modes, which allows for size selection and isolates nucleic acid molecules from other molecules. In some embodiments, in addition to drawing a molecule into a well, reversal of polarity of the electric field can expel the molecule from the well in which sensing occurs, thereby making the molecule sensing apparatus 100 reusable.

One of the features of the molecule sensing apparatus 100 is a zero-mode waveguide (ZMW), which can be generally considered the “active” wells, where the term “active” refers to the electric field that is produced in the wells by a combination of the electrically conductive layer that forms a portion of the well and the electrically conductive element that is disposed above an entrance to the well. Advantages of the ZMW format disclosed herein over previous versions include: 1) high capture efficiency (orders of magnitude) over ordinary ZMWs, 2) direct deposition on solid-substrates, which makes these more mechanically stable than previous-generations of nanopore ZMWs (NZMWs) and porous-layer ZMWs (PZMWs), 3) higher-throughput than NZMWs and PZMWs, because no freestanding membrane is required, higher signal-to-background ratio than membrane-containing ZMWs (NZMWs and PZMWs), 4) reduced fabrication costs, 5) more accurate radial positioning of the enzyme probed in a single-molecule assay, 6) longer lifetime of devices than membrane-containing ZMWs (NZMWs and PZMWs).

The critical step in DNA loading for sequencing is capture and binding of a DNA fragment into a zero-mode waveguide (ZMW), which is an about 100 nm cylindrical well in which a sequencing reaction takes place. Since long DNA fragments (>1,000 bp) are longer than 100 nm, passive entry of these long molecules into ZMWs is not straightforward. Earlier nanopore zero-mode waveguides (NZMWs) were employed as tools to draw DNA fragments electrokinetically into the ZMWs. Such devices are capable of drawing pg-levels of DNA. Since NZMW manufacture is difficult to scale up, however, described herein is a scalable process for depositing a porous ceramic layer in order to draw DNA into the ZMW wells.

Surprisingly, these porous zero-mode waveguides (PZMWs) are showing a lot of promise because they allow capture of DNA and RNA from even lower input levels, about one order of magnitude better than NZMWs, and six to seven orders of magnitude better than commercial ZMWs used in PacBio® SMRT sequencing. In addition to a highly efficient capture, the PZMW's that capture DNA fragments without length-bias, which means that long DNAs are captured with similar efficiency as short DNAs. This is useful for achieving native DNA sequencing from single-cell inputs because there are so few DNA fragments to read if DNA amplification is not to be used. However, the porous ZMWs require a freestanding ultrathin membrane to be used, which compromises stability of resulting devices, as well as reducing the signal-to-noise of fluorescence measurements.

FIG. 1C is a schematic diagram of electronics used to sense molecules in the molecule sensing chamber 101 of FIG. 1A in an automated manner. Referring to FIG. 1C, the electronics includes a controller 191, the power source 136, and the molecule sensor 190. The controller 191 is communicatively coupled to the power source 136 and molecule sensor 190 to coordinate actions for automatically sensing the molecules in the molecule sensing apparatus 100 of FIGS. 1A-B. Letter designations in FIG. 1C, letters A-G, indicate an example sequence in which the automatic sensing of the molecules may occur.

In the example sequence of FIG. 1C, in Step A, the controller 191 provides an instruction to the power source 136 to capture (or release) a molecule in the wells by sending a capture/release molecule signal 161 to the power source 136. Responsively, in Step B, the power source 136 outputs voltages V₁ and V₂ (DC, AC, or other signal waveform) on respective wires (not shown) of the power source cable 137. After the power source 136 has output the voltages in Step B, in Step C, the power source 136 sends an acknowledgement signal 162 to the controller 191 that the power has been delivered to the molecule sensing chamber 101, whereby V₂ is delivered to the electrically conductive layer 110 and V₁ is delivered to the electrically conductive element 135.

After the power has been delivered, in Step D, the controller 191 issues a ‘begin sensing’ signal 192 to the molecule sensor 190. Responsively, in Step E, the molecule sensor 190 transmits a sensor signal 196, which may be an optical signal in the visible or near infrared region, via the molecule sensor cable 103. In Step F, a sensed signal 197 obtained through observation of the wells for the molecule is returned to the molecule sensor 190. The molecule sensor 190 thereafter may, in step G, return a representation of molecule(s) 193 to the controller 191. The controller 191 may thereafter transmit the representation of the molecules 193 via a wired, fiber-optic, or wireless communications channel (not shown) to a device (not shown) accessible to the user to provide information to the user 90 about the molecules sensed.

As should be understood, after the power source 136 is activated, there may be a time lag between the activation and the molecules' entering the wells for observation by the molecule sensor 190. The controller 191, therefore, may be programmed to delay activation of the molecule sensor 190 such that a valid sensing of the molecules is performed. Thus, the automated measuring process of Steps A-G may take minutes or hours, depending on how quickly a molecule enters a well for observation.

Presented immediately below are further example embodiments, followed by more details beginning at FIG. 2A.

In an embodiment, an apparatus for sensing a molecule comprises a substrate layer, a sample interface layer, and an electrically conductive layer. The sample interface layer has a sample interface side and a substrate layer facing side. The electrically conductive layer is disposed between the substrate layer facing side of the sample interface layer and the substrate layer. The sample interface layer and the electrically conductive layer each define a respective opening therethrough that, aligned, compose a wall of a well with a bottom boundary defined by the substrate layer. The electrically conductive layer, when energized with a given polarity relative to an electrically conductive element in a sample at the sample interface layer, produces an electric field from the electrically conductive layer through the well to the electrically conductive element. The electric field is sufficient to draw a molecule, of polarity opposite from the given polarity, from the sample at the sample interface side into the well toward the electrically conductive layer.

In another embodiment, the apparatus further comprises a voltage potential source configured to apply a voltage potential difference between the electrically conductive element and the electrically conductive layer. In another embodiment, the substrate layer is a transparent material at visible and the near infrared wavelengths, and the sample interface layer is an optically reflective layer at the visible and the near-infrared wavelengths.

In an embodiment, the substrate layer further comprises an optical sensor system. The optical sensor system has an arrangement to direct a wavelength to the well via the substrate layer and collect a response from the molecule via the substrate layer, and wherein the electrically conductive layer, through its optically reflective property, limits the transmission of wavelengths to the sample layer above the interface layer. Optical noise is due to the excitation of the sample. If the sample interface layer is not reflective, then all fluorescent molecules in the sample above the interface layer become excited by incoming wavelengths through the substrate and emit light, causing background noise. In some embodiments, the sample interface layer, the electrically conductive layer, and the substrate layer define multiple wells. The sensor system is configured to sense a respective molecule in the multiple wells in a parallel manner.

The visible wavelengths range from about 400 nm to about 800 nm. The near-infrared wavelengths range from about 800 nm to about 2,500 nm. The well is a zero-mode waveguide relative to the visible wavelengths. The wells may be cylindrically shaped holes of about 10 nm to about 150 nm in diameter and at least 100 nm in length (i.e., depth). The transparent material may be made of fused silica, quartz, or glass.

In another embodiment, the sample interface layer is a metal. In this embodiment, the apparatus further comprises an electrically non-conductive layer positioned between the sample interface layer and the electrically conductive layer, wherein the electrically non-conductive layer defines a respective opening aligned with the opening of the sample interface layer and the opening of the electrically conductive layer. The electrically non-conductive layer may be made of a material including silicon dioxide, aluminum oxide, or silicon nitride. The metal may include platinum, gold, silver, titanium, aluminum, palladium, iridium oxide, palladium that can be applied to gold electrodes, iridium oxide that can be applied to gold electrodes, or a combination thereof.

FIG. 2A depicts a molecule sensing apparatus 200 having a sample interface layer 220, an electrically nonconductive layer 215, an electrically conductive layer 210, and a substrate layer 205, in accordance with an embodiment of the invention. The sample interface layer 220 has a sample interface side 217 and a substrate layer facing side 216. The electrically conductive layer 210 is disposed between the substrate layer facing side 216 of the sample interface layer 220 and the substrate layer 217. The sample interface layer 220 and the electrically conductive layer 210 each define a respective entrance 250 therethrough that, aligned, compose a wall 230 of a well 225 with a bottom boundary defined by the substrate layer 205. The electrically conductive layer 210, when energized with a given voltage V₂ 247, results in a given polarity relative to another voltage V₁ 248 of an electrically conductive element 235 in a sample at the sample interface layer 220, produces an electric field 241 from the electrically conductive layer 210 through the well 225 to the electrically conductive element 235. The electric field 241 is sufficient to draw a molecule 255 of a charge 256 from the given polarity of the electrically conductive layer 210, in a direction 242 from the sample at the sample interface side 217 through the well 225 toward the electrically conductive layer 210.

FIG. 2B depicts the molecule sensing apparatus 200 of FIG. 2A having a sample interface layer 220, an electrically nonconductive layer 215, an electrically conductive layer 210, to which a primer-enzyme complex 271 is coupled, and a substrate 205, in accordance with an embodiment of the invention. In an embodiment of the invention, the sample interface layer 220 has a sample interface side 217 and a substrate layer facing side 216. The electrically conductive layer 210 is disposed between the substrate layer facing side 216 of the sample interface layer and the substrate layer 217. The sample interface layer 205 and the electrically conductive layer 210 each define a respective opening therethrough that, aligned, compose a wall 230 of a well 225 with a bottom boundary defined by the substrate layer 205. The electrically conductive layer 210, when energized with a given voltage V₂ 247 relative to an electrically conductive element 235 in molecule sensing system containing a sample 121, as depicted in FIG. 1A-B, at the sample interface layer 220, produces an electric field 241 from the electrically conductive layer 210 through the opening to the electrically conductive element 235. In some embodiments, the electrically conductive layer 210 can have a positive charge 258. In some embodiments, the electrically conductive element 235 can have a negative charge 257. In some embodiments, the molecule 255 has a negative charge 256. The electric field 241 is sufficient to draw a molecule 255, of a polarity 256 opposite to the given polarity, from the sample at the sample interface side 217 from the entrance 250 through the well 225 toward the electrically conductive layer 210.

FIG. 2B further depicts an organic matter 265, fixedly located in the well 225, that is selected based on a property that enables the molecule 255 to couple chemically thereto. In another embodiment, the molecule 255 is DNA or RNA and the organic matter 265 is an enzyme that upon binding molecule 255 forms a complex 271. The DNA or RNA can consist of single-stranded DNA (ssDgNA), double stranded DNA (dsDNA), single-stranded RNA (ssRNA) and double-stranded RNA (dsRNA). In this example embodiment, the organic matter 265 is attached to an electrically nonconductive layer 215 in the well 225 through biotinylated glycol silane-based functionalization of the substrate layer 205. In another embodiment, the streptavidin moiety 270 is replaced with a biotinylated primer that can bind to nonconductive layer 215.

In some embodiments, the well forms the shape of a cylinder, truncated cone, or any polygonal prism. In yet another embodiment, the entrance at the sample interface side of the sample interface layer may have a diameter from about 20 nm to about 250 nm.

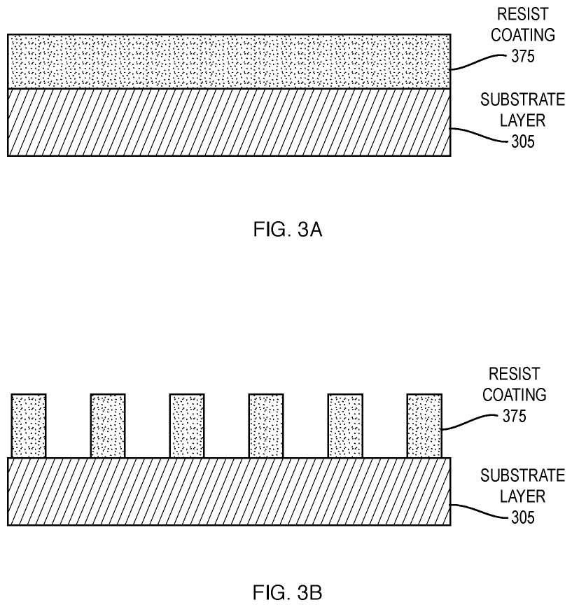

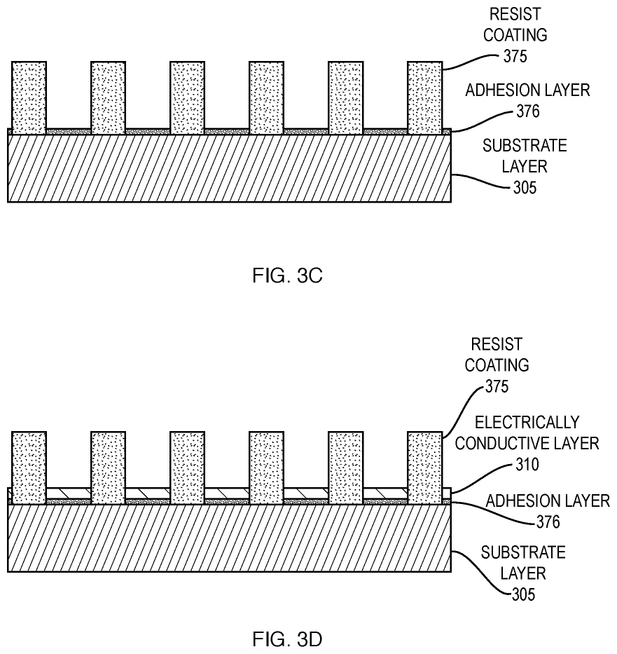

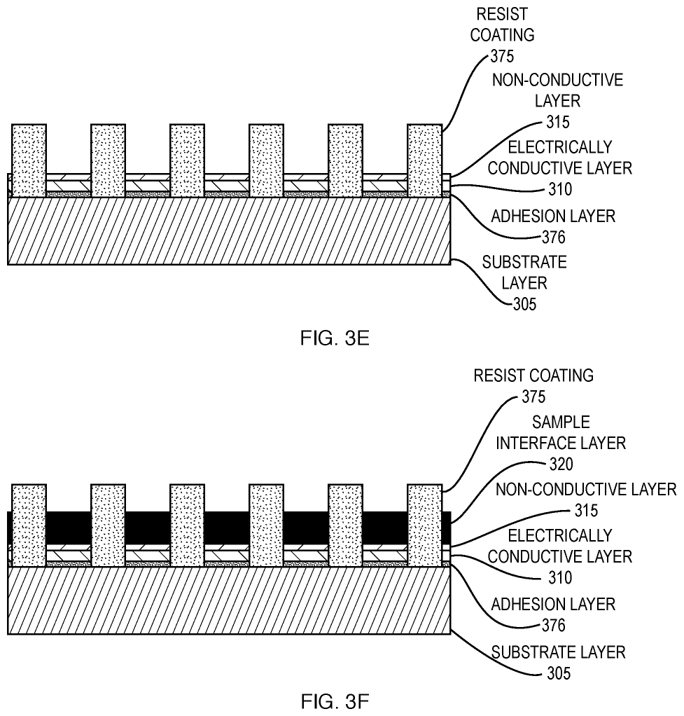

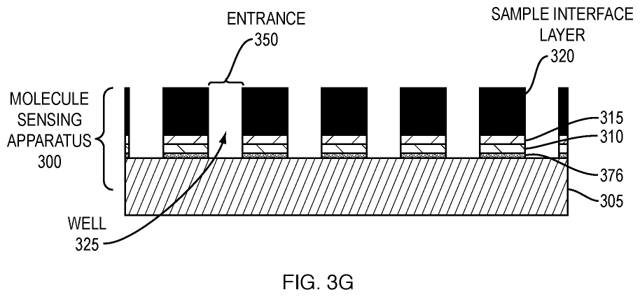

FIG. 3A-3G are a sequence of side views of a structure that illustrates the manufacturing of a molecule sensing apparatus 300 for detecting a molecule 255, described in reference to FIG. 2, in accordance with an embodiment of the invention.

Referring first to FIG. 3A, in an embodiment, a method of manufacturing a molecule sensing apparatus 300 for detecting a molecule comprises applying an electron beam lithography resist coating 375 to a substrate layer 305, removing portions of the electron beam lithography resist coating 375 to define a pattern of support elements composed of the electron beam lithography resist coating 375.

As seen in FIGS. 3B-F, an adhesion layer 376 is applied to the substrate layer 305, which facilitates the coupling of the electrically conductive layer 310. The electrically conductive layer 310 is applied above the substrate layer 305 within the pattern of support elements. A sample interface layer 320 is then applied above the electrically conductive layer 310 within the pattern of support elements. In some embodiments, an adhesion layer 376 facilitates the coupling of the electrically conductive layer 310 to the substrate layer 305. In some embodiments, a nonconductive layer 315 is applied between the electrically conductive layer 310 and the sample interface layer 320.

FIG. 3G is a side view of a manufactured molecule sensing apparatus 300 for detecting a molecule, in accordance with an embodiment of the invention. Removing the electron beam lithography resist coating 375 support elements creates entrances 350 of wells 325 having a boundary defined by the electrically conductive layer 310 and the sample interface layer 320. In another embodiment, the method forms an electrically non-conductive layer 315 above the electrically conductive layer 310 within the pattern of support elements.

In an embodiment, a system for sensing a molecule comprises a structure, a power supply, and a molecule sensor. The structure includes a sample interface layer, an electrically conductive layer, and a substrate layer, arranged in that order. The sample interface layer and electrically conductive layer define openings therethrough that are aligned to form a well from a sample interface side of the sample interface layer to the substrate layer. The power supply is coupled to the electrically conductive layer and at least one electrically conductive element. The at least one electrically conductive element located external from the well, the electrically conductive layer and the at least one electrically conductive element, when powered relative to each other, produce an electric field in the well of sufficient strength to cause a molecule, from a sample at the sample interface side of the sample interface layer, with a polarity opposite a polarity of the electrically conductive layer to travel via the well toward the electrically conductive layer. The molecule sensor is communicatively coupled to the structure in an arrangement that enables sensing of the molecule in the well. In another embodiment, a controller may be configured to enable the power supply to deliver a voltage difference to the electrically conductive layer and at least one electrically conductive element and to provide an indicator signal to the molecule sensor to notify the molecule sensor to perform a sensing of the molecule.

In another embodiment, a controller is configured to change a voltage level applied to the electrically conductive layer and the electrically conductive element to a potential difference sufficient to enable the molecule to exit the well. In another embodiment, the power source produces a voltage waveform that has a root-mean-square (rms) voltage level sufficient to draw molecules to the wells based on their electric charge or their polarizability. In another embodiment, the molecule sensor is configured to produce a representation of a presence of the molecule or of the molecule itself.

In an embodiment, a method of sensing a molecule comprises applying an electric field to a sample in proximity of a structure defining a well and optically sensing the molecule within the well via intrinsic or externally-induced fluorescence. The electric field applied to the sample is of sufficient strength to cause a molecule of a given polarity in the sample to be drawn toward a surface in the well of an opposite polarity.

In an embodiment, a method for fabricating lasso-containing ZMWs are typically cylindrical shaped holes, such as 100 nm to 150 nm in diameter and 100 nm long. The method can be broken down to the following steps:
1) A negative electron beam lithography resist is spin-coated on a wafer and baked on a hotplate.
2) ZMW pattern and alignment markers are exposed using an e-beam lithography. The wafer is developed leaving behind an array of pillars on the substrate.
3) A titanium layer of few nanometers is evaporated on the substrate as to promote adhesion
4) A thin layer of metallic film is deposited over the adhesion film, which will serve as the electrode.
5) A dielectric material is deposited on top of the electrode layer to insulate the metallic electrode from the cladding layer.
6) 100 nm cladding layer is deposited (MZMW).
7) A lift-off process is carried out by sonicating the wafer in a solvent, other thickness, such as +/−5 nm, +/−10 nm, +/−25 nm, may alternatively be deposited, which leaves behind the ZMW structure after breaking off the aluminum heads at the top of the e-beam resist and dissolving the resist pillars.

Pg-level DNA and RNA can be captured into the ZMWs by simply applying a voltage. This is novel and not the case with existing technology.

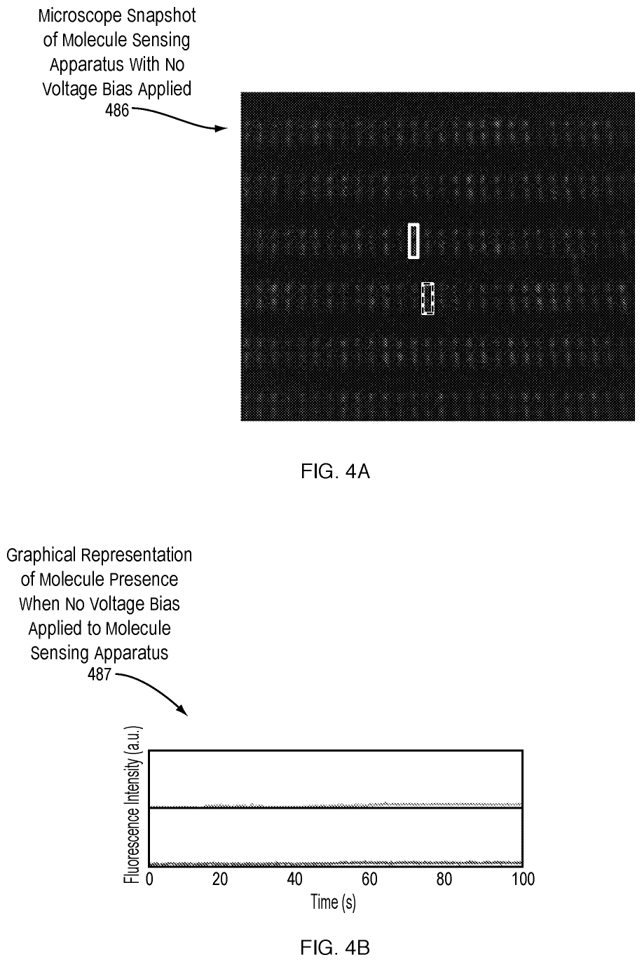

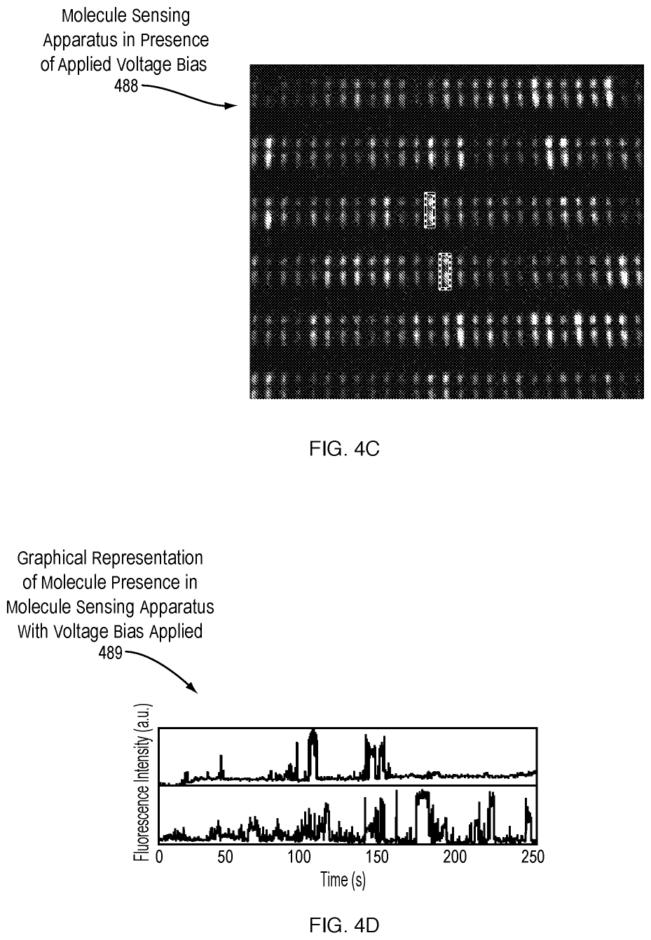

FIGS. 4A-B depict a molecule sensing apparatus to which a voltage has not been applied, in accordance with an embodiment of the invention. A molecule sensing apparatus with no applied voltage 486. A graphical depiction of fluorescence 487 corresponding to molecule presence in a sample reveals that when no electric field is applied, molecule presence if virtually non-existent. In the presence of a supporting electrolyte (e.g., 10 mM KCl buffer, pH8), application of a voltage to the lasso results in an ion current that leads to electrokinetic focusing of DNA inside the ZMWs. To demonstrate DNA capture using electrochemical lassos, a 50 nM solution of 2.5 kbp dsDNA, labeled with DiYO-1 dye mixed at 10:1 basepair:dye ratio was used, and the change in fluorescence intensity at ZMWs was recorded using an EMCCD camera, before and after applying a voltage. The bright spots that appear after applying voltage in FIG. 4b demonstrate entry of labeled DNAs into the wells. The DNA capture can be better seen in a continuous optical recording of ZMWs (ZMWs marked by blue and purple boxes in FIG. 3a) before and after applied voltage. In the absence of an applied voltage, no activity is observed, indicating poor diffusion-based DNA capture in the ZMWs.

FIG. 4C-D depict a molecule sensing apparatus to which a voltage is applied 488. A graphical depiction of fluorescence 489 of a molecule present in a sample demonstrates that when a voltage is applied, detection of a molecule in a sample is significantly increased. Upon application of a voltage (−1V), DiYO-1-stained DNAs enter the illumination volume of ZMWs and get excited by a blue laser. The fluorescence bursts visible in these optical traces indicate entry of individual DNA molecules into the marked ZMWs. This method can be used to capture RNA, proteins, and other molecules for interrogation inside the ZMWs. DNA capture can be done in any buffers, as long as an adequate electric field is applied to the lasso. Moreover, choice of the metallic electrode and addition of a redox couple to the buffer can further facilitate the capture. These devices were used to determine the sequence of a 260 bp DNA template, loaded to ZMWs at sub-nanogram levels. A complex of DNA-primer-polymerase was made and added to the ZMWs along with the fluorescently labeled dNTPs, so that the final concentration of DNA templates and the dNTPs are 1 nM and 50 nM, respectively. The template DNA was immobilized on the biotinylated ZMW floor. We then initiated the enzyme activity by adding a solution containing Mg²⁺, which activates the polymerase. Immediately, discrete fluorescence bursts were observed indicating base incorporations into growing DNA strands.

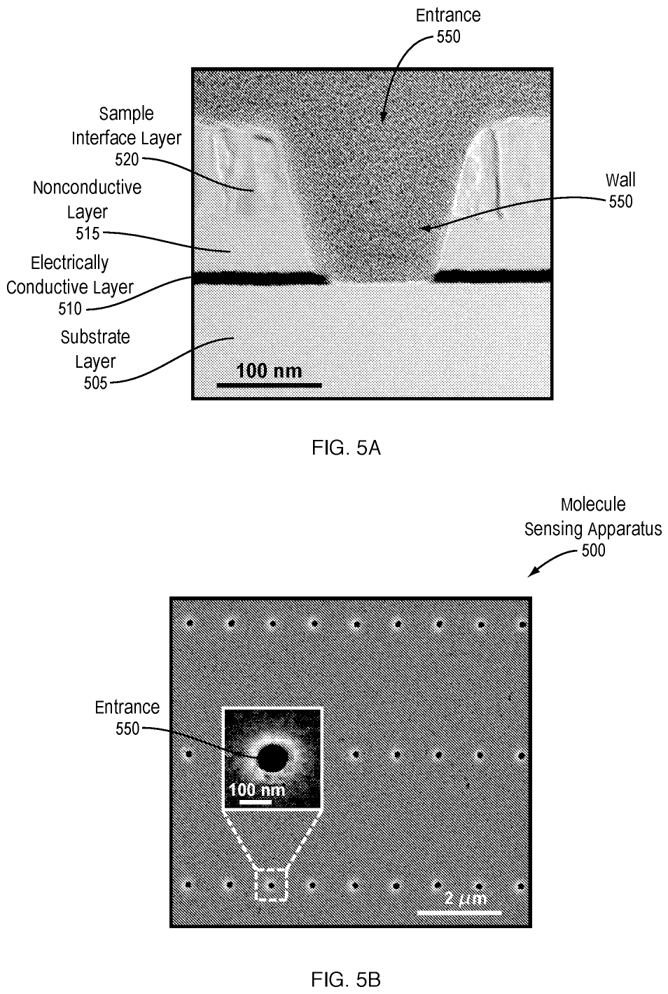

FIG. 5A is a side view of a well and layers defining the well, in accordance with an embodiment of the invention. FIG. 5A illustrates an entrance 550 to a well 525 having a sample interface layer 520, an electrically conductive layer 510, and a substrate layer 505. In some embodiments, a nonconductive layer 515 is located between the sample interface layer 520 and the electrically conductive layer 510. FIG. 5B is a top view of a molecule sensing apparatus, in accordance with an embodiment of the invention. FIG. 5B illustrates the orientation of entrances 550 into areas that are depicted in FIG. 5A as wells 525.

In some embodiments, an alternative system for capturing DNA molecules into ZMWs that does not require the use of a freestanding membrane. The working principle of the system relies on embedding metallic electrodes under the waveguides to create an electric field. Application of a voltage to the electrode layer with the use of proper electrolyte allows efficient electrophoretic DNA capture at picogram levels. To do so, a thin metallic film (M_{Lasso}) is deposited on a substrate (S) which serves as an electrode. This electrode is insulated from a metallic ZMW layer (M_{ZMW}) using a dielectric spacer (D). This device eliminates the need for free-standing membranes, and therefore is mechanically stable, allows scaled-up fabrication, reduces the background optical noise, and improves the DNA loading efficiency by several orders of magnitude. Dual-ring gold-based zero-mode waveguides have been previously used in the context of electrochemical analysis, wherein electrochemical signals are amplified by a repetition of reduction and oxidation reaction at two closely located electrodes. However, the system disclosed herein has never been used for DNA/RNA sequencing and biosensing applications.

An alternative approach for making these ZMWs is to deposit the layers in steps 3-6, spin coat a positive e-beam resist, and then expose hole arrays using e-beam lithography, and finally, ion-mill the cavities and remove the e-beam resist layer. An advantage of this method is that defect pinholes are avoided by eliminating the lift-off process that is essential when using a negative e-beam resist.

Glass or fused silica can serve as the substrate (S) in this device. The cladding layer of ZMWs is usually opaque and/or reflective material which insulates penetration of the electric and the magnetic fields. The cladding layer material (M_{ZMW}) choices include, but are not limited to, aluminum, copper, gold, silver, chromium, platinum, titanium, etc. A comparison of single-molecule emission indicates that gold ZMWs are more suitable than aluminum ZMWs for single molecule fluorescence studies in the red region of the visible spectrum. On the other hand, aluminum is better suited for the green region. This layer is insulated by a dielectric, such as aluminum oxide, silicon dioxide, or silicon nitride from the electrode layer which can be made of platinum, silver, gold, or palladium (M_{Lasso}).

### Alternative Designs

Alternative ZMW structures can aid to accurately position the polymerase in the wells, facilitate fabrication, and further improve the capture efficiency. In some embodiments, the choice of silicon dioxide as the dielectric layer, allows tethering the DNA-polymerase to this layer through biotinylated polyethylene glycol silane-based functionalization of a SiO₂ layer, represented in FIG. 2A-B as a substrate layer 205. In those embodiments, polymerase molecules will be placed in the same relative position in all the wells owing to the symmetry of the ZMW structures, which improves consistency in the signal to noise ratios from one ZMW to another. In some embodiments, polymerase immobilization at the floor of the ZMWs will be inhibited by coating the wafers with a thin base layer HfO₂, prior to the fabrication of the e-beam resist pillars. This layer allows selective chemical functionalization using poly-vinyl-polyphosphonate molecules, which prevent non-specific binding of organic molecules to the surface. Furthermore, in order to facilitate the fabrication, the lasso can serve as the ZMW cladding, too. In some embodiments, a metal layer serving as both lasso and cladding is coated with a dielectric material (such as Al₂O₃) to prevent short-circuiting with the buffer above.

In some embodiments, is an alternative format in which a porous layer (P) is deposited between the electrode layer (M_{Lasso}), represented in FIG. 2A-B as the electrically conductive layer 210, and the dielectric layer (D), represented in FIG. 2A-B as the nonconductive layer 215, which serves to enhance the currents obtained from voltage application across the ZMWs, represented in FIG. 2A-B as the sample interface layer 220, by having a greater contact area with ions in the electrolyte. The reverse could also be done, in which the porous layer is deposited before the electrode layer (both configurations are expected to yield similar results).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. An apparatus for sensing a molecule, the structure comprising:
a substrate layer;
a sample interface layer having a sample interface side and a substrate layer facing side;
an electrically conductive layer disposed between the substrate layer facing side of the sample interface layer and the substrate layer; and
wherein the sample interface layer and electrically conductive layer each define a respective opening therethrough that, aligned, compose a wall of a well with a bottom boundary defined by the substrate layer, the electrically conductive layer, when energized with a given polarity relative to an electrically conductive element in a sample at the sample interface layer, produces an electric field from the electrically conductive layer through the well to the electrically conductive element, the electric field sufficient to draw a molecule, of polarity opposite from the given polarity, from the sample at the sample interface side through the well toward the electrically conductive layer.

2. The apparatus of claim 1, further comprising a voltage potential source configured to apply a voltage potential difference between the electrically conductive element and the electrically conductive layer.

3. The apparatus of claim 1, wherein the substrate layer is a transparent material at visible and near-infrared wavelengths and the sample interface layer is an optically reflective layer for the visible and near-infrared wavelengths; and further comprising an optical sensor system, the optical sensor system having an arrangement to direct a wavelength to the well via the substrate layer and collect a response from the molecule via the substrate layer, the optically reflective layer limiting transmission of the wavelengths to the sample above the interface layer.

4. The apparatus of claim 3, wherein the sample interface layer, electrically conductive layer, and substrate layer define multiple wells, and wherein the sensor system is configured to sense a respective molecule in the multiple wells in a parallel manner.

5. The apparatus of claim 3, wherein the visible wavelengths range from about 400 nm to about 800 nm, and wherein the well is a zero-mode waveguide relative to the visible wavelengths.

6. The apparatus of claim 3, wherein the wells are cylindrically shaped holes of about 100 nm to about 150 nm in diameter and at least 100 nm in length.

7. The apparatus of claim 3, wherein the transparent material includes fused silica, quartz, or glass.

8. The apparatus of claim 1, wherein the sample interface layer is a metal, and further comprising an electrically non-conductive layer positioned between the sample interface layer and the electrically conductive layer, wherein the electrically non-conductive layer defines a respective opening aligned with the opening of the sample interface layer and the opening of the electrically conductive layer.

9. The apparatus of claim 8, wherein the electrically non-conductive layer is made of a low-dielectric material including silicon dioxide, aluminum oxide, or silicon nitride.

10. The apparatus of claim 8, wherein the metal includes platinum, gold, silver, titanium, aluminum, or combination thereof.

11. The apparatus of claim 1, further comprising an organic matter, fixedly located in the well, that is selected based on a property that enables the molecule to chemically couple thereto.

12. The apparatus of claim 11, wherein the molecule is DNA or RNA, and wherein the organic matter is a complex comprising a DNA or RNA-processing enzyme to which the molecule binds.

13. The apparatus of claim 11, wherein the organic matter is attached to an electrically non-conductive layer in the well through biotinylated polyethylene glycol silane-based functionalization of the substrate layer.

14. The apparatus of claim 1, wherein the well forms the shape of a cylinder, truncated cone, or any polygonal prism.

15. The apparatus of claim 1, wherein the opening at the sample interface side of the sample interface layer, the opening having a diameter from about 20 nm to about 250 nm.

16. A method of manufacturing an apparatus for detecting a molecule, the method comprising:
applying a negative electron beam lithography resist coating to a substrate layer;
removing portions of the electron beam lithography resist coating to define a pattern of support elements;
forming an electrically conductive layer above the substrate layer within the pattern of support elements;
forming a sample interface layer above the electrically conductive layer within the pattern of support elements; and
removing the support elements to create wells having a boundary defined by the electrically conductive layer, the electrically conductive layer and the sample interface layer.

17. The method of claim 16, further comprising forming an electrically non-conductive layer above the electrically conductive layer within the pattern of support elements.

18. A system for sensing a molecule, the system comprising:
a structure including a sample interface layer, an electrically conductive layer, and a substrate layer arranged in that order, the sample interface layer and electrically conductive layer defining openings therethrough that are aligned to form a well from a sample interface side of the sample interface layer to the substrate layer;
a power supply coupled to the electrically conductive layer and at least one electrically conductive element, the at least one electrically conductive element located external from the well, the electrically conductive layer and the at least one electrically conductive element, when powered relative to each other, produce an electric field in the well of sufficient strength to cause a molecule, from a sample at the sample interface side of the sample interface layer, with a polarity opposite a polarity of the electrically conductive layer to travel via the well toward the electrically conductive layer;
a molecule sensor that is communicatively coupled to the structure in an arrangement that enables sensing of the molecule in the well; and
a controller configured to enable the power supply to deliver a voltage difference to the electrically conductive layer and at least one electrically conductive element and to provide an indicator signal to the molecule sensor to notify the molecule sensor to perform a sensing of the molecule.

19. The system of claim 18, wherein the controller is configured to change a voltage level applied to the electrically conductive layer and the electrically conductive element to a potential difference sufficient to enable the molecule to exit the well.

20. The system of claim 18, wherein the power source produces a voltage waveform that has a root-mean-square (rms) voltage level sufficient to draw molecules to the wells based on their electric charge or their polarizability.

21. The system of claim 18, wherein the molecule sensor is configured to produce a representation of a presence of the molecule or of the molecule itself.

22. A method of sensing a molecule, the method comprising:
applying an electric field to a sample in proximity of a structure defining a well, the electric field of sufficient strength to cause a molecule of a given polarity in the sample to be drawn toward a surface in the well of an opposite polarity; and
optically sensing the molecule in the well via intrinsic or externally-induced fluorescence.
